(19) United States
(12) Patent Application Publication
(10) Pub. No.: US 2025/0381035 A1
(43) Pub. Date: Dec. 18, 2025

(54) COAPTATION DEVICE WITH POSITIONING SYSTEM

(71) Applicant: SINGAPORE HEALTH SERVICES PTE LTD, Singapore (SG)

(72) Inventor: James R. Watson, Santa Rosa, CA (US)

(21) Appl. No.: 18/877,670

(22) PCT Filed: Jun. 28, 2023
(86) PCT No.: PCT/US2023/069296

§ 371 Date: Dec. 20, 2024

Related U.S. Application Data
(60) Provisional application No. 63/367,198, filed on Jun. 28, 2022.

(51) Int. Cl. A61F 2/24

(52) U.S. Cl. CPC A61F 2/246; A61F 2/2466; A61F 2220/0008; A61F 2220/0025

(57) ABSTRACT

An implantable prosthesis and delivery' system for treating tricuspid valve regurgitation (TVR). The system is configured for pre-loading into a percutaneous delivery 7 system. The system includes a self-expanding anchoring stem with an attached and positionable coaptation member. The stem is implanted in the inferior vena cava proximate the right atrium and is connected to the coaptation member with a. steering tube and a multi-directional coupler and gimbal assembly. The coaptation member is fabricated from a porous or semi-porous material formed over a wire frame and is configured, possibly with leaflet matching curvature, before implantation. When deployed, the coaptation member self-aligns, self-inflates, and takes shape over several cardiac cycles to conform to the patient's TV defects and to provide coaptation surfaces for native leaflets to reduce TVR.

### BACKGROUND OF THE INVENTION

### Technical Field

The present invention relates most generally to medical procedures and devices for repairing damaged or diseased valves, and more particularly a tricuspid valve prosthesis with a delivery system that enables precise positioning and placement during implantation and deployment to effectively provide a high efficiency coaptation surface in the treatment of tricuspid regurgitation (TR) in diverse patient anatomies.

### Background Discussion

The tricuspid valve (TV) comprises multiple arrangements of native tissue leaflets and a corresponding circumferential tissue ring (annulus) within the right heart structure. The inferior vena cava (IVC) returns de-oxygenated blood to the right atrium (RA) for subsequent flow through the TV into the right ventricle (RV) and eventually to the lungs for reoxygenation. In TVR, the tricuspid valve between the right atrium and the right ventricle, does not close properly after blood is pumped from the right atrium into the right ventricle. Improper coaptation between the native leaflets (anterior, posterior, and septal) may result from several causes, including enlargement of the TV annulus, structural damage to the chordae tendineae, papillary muscle compromise, and so forth. As a result of the improper coaptation, at high ventricular contraction pressures during ventricular systole, blood flows back from the right ventricle into the right atrium.

Designing medical devices that effectively reduce TR in patients is a challenging problem. When pharmacological interventions such as diuretics or vasodilators are ineffective, there remain two primary solutions at present: (1) a mechanical solution to remodel the TV annulus shape and size to force the leaflets closer together for coaptation, which increases the risk to fragile tissue; and (2) the insertion of a device that “closes the gap” to prevent or reduce TR in the TV via coaptation with the native leaflets.

The designs presented in this application are directed to the latter type of solution, wherein a novel and improved prosthesis, viz., a coaptation device, is safely anchored in the IVC and easily positioned within the TV leaflets using a novel delivery system that enables multi-directional position and placement of the coaptation device in an optimal location within the TV. The inventive delivery system and the coaptation prosthesis it delivers is unique in its freedom of movement in multiple axes to allow unencumbered contact with the native leaflets, and thus to avoid giving rise to new TR.

### DISCLOSURE OF INVENTION

In its most essential aspect, the present invention is an implantable prosthesis and delivery system for treating TVR. A prothesis configured for pre-loading into a percutaneous, co-axial, over-the-wire delivery system. It includes a self-expanding anchoring stent with an attached and positionable coaptation member, referred to herein as a coaptation sail.

The stent is implanted in the inferior vena cava proximate the right atrium and is connected to the coaptation sail with a steering tube. The coaptation sail is fabricated from a porous or semi-porous material and is configured before implantation and deployment to closely match patient anatomy and the morphological type of TVR to be treated (e.g., leaflet damage, annular dilation, or patterns of right hear remodeling). When deployed, the coaptation sail conforms to the patient valve defects over several cardiac cycles by self-aligning in the TV, absorbing blood, sequestering absorbed blood from turbulent blood flow, allowing a volume of such absorbed blood to coagulate and undergo mechanical deformations under contacts with the leaflets, and thereby eventually assuming a size and shape that fills the coaptation gap addressed and provide coaptation surfaces for the native TV leaflets.

A primary component of the prosthetic system is the coaptation sail. In embodiments it may be fabricated from medical grade surgical fabric sewn over a wire frame, which is permanently attached to a steering tube. The fabric covered sail frame extends into the tricuspid valve (TV) to provide a coaptation structure for the native TV leaflets. Materials other than surgical fabric may be employed, including several porous, semi-porous, and even non-porous materials, such as a woven fabric or a polymer barrier. Several types of open-cell medical grade foams may also be employed, including polyurethane foam (PU), reticulated polyurethane, polytetrafluoroethylene (PTFE), etc.

In embodiments, a coupler permanently connects the coaptation member (coaptation sail) to the steering tube and allows multi-axial rotational movement of the sail to enable self-aligning with the native leaflet coaptation lines and commissures. The steering tube is itself an adjustable member that responds to tensioning inputs (i.e., a tightening of an internal structure) that rotates the coaptation sail towards the TV to aid in positioning the coaptation sail within the native TV annulus. The steering tube is connected to the delivery system handle to enable coaptation sail positioning.

An anchor securely locks the adjusted steering tube position during an implantation procedure; and it also disconnects to enable delivery system removal. A stent is positioned in the IVC, near the juncture of the right atrium (RA) and IVC, and provides anchoring of the coaptation prosthesis.

Deliberate control inputs during an implantation procedure are made through a delivery system handle, which provides multiple functions during the preparation and implantation of inventive prosthesis. The control system handle enables a finely controlled implantation and complete retrieval of the prosthesis, if needed.

A flush-port in the handle facilitates flushing the system with heparinized saline to remove all air from the inner catheter and prosthesis.

A sheath dial on the control handle retracts the outer sheath upon rotation and slowly exposes the prosthesis and stent.

A heparinized saline drip line through the control system handle promotes non-coagulation of the adjustment mechanisms during prosthesis delivery, and a stent release button prevents accidental prosthesis release until it is pushed by preventing the outer sheath from fully retracting.

A tension knob on the proximal end of the handle adjusts the amount of tension applied to the prosthesis upon rotation, and a release button disconnects the delivery system from the prosthesis.

### BRIEF DESCRIPTION OF THE DRAWINGS

FIG. 1 is a perspective view of the inventive tricuspid valve prosthesis and steering system subassembly of the present invention;

FIG. 2 is a front view in elevation of the control handle for the prosthesis delivery system;

FIGS. 3A-3C are perspective views of the distal end of the steering tube, gimbal, and coupler assembly, including the nitinol wires of the coaptation sail, with FIG. 3A being an upper front view, FIG. 3B being an exploded view thereof, and FIG. 3C being a side view showing the components assembled and operatively connected;

FIGS. 4A-4C are perspective views showing several orientations of one steering system at various flexure amounts and rotations, thereby highlighting its degrees of freedom and ranges of motion for positioning the placing the coaptation sail in the TV;

FIG. 5 is a detailed view of the stent and steering system subassembly of the inventive delivery system and coaptation prosthesis device, here featuring the connection between the stent and the steering tube subassembly;

FIG. 6 is an upper perspective view showing the tensioning rod subassembly coupled to the steering system subassembly;

FIGS. 7A-7C are perspective views of the tensioning rod assembly shown in FIG. 6, FIG. 7A being an assembly view, FIG. 7B being an exploded view, and FIG. 7C being a cross-sectional view thereof;

FIG. 8 is detailed exploded view of select components of the tensioning rod subassembly shown in FIGS. 7A-7C;

FIG. 9 is a detailed cross-sectional view of the steering system and tensioning rod subassemblies taken along section lines 9-9 of FIG. 5;

FIG. 10 is a detailed view of attachment structure for connecting the steering tube to the stent;

FIG. 11 is a more detailed view of the steering tube and tensioning rod subassemblies shown in FIG. 9;

FIGS. 12A-12C are perspective views showing details of an alternative tensioning member locking mechanism, with FIG. 12A being an exploded view, FIG. 12B being a detailed view showing details from window 12B of FIG. 12A, and FIG. 12C showing details, with detailed views incorporated, showing an alternative tensioning member locking mechanism;

FIGS. 13A-13C are perspective views of the distal end of the delivery system control handle, FIG. 13A being an assembly view, FIG. 13B being a cross-sectional view, and FIG. 13C being an assembly view showing the tension knob extended; and

FIG. 14 is a highly schematic cross-sectional view of the coaptation sail aligned in the TV and the anchoring stent in the IVC, the two components connected by the steering tube, all in accordance with embodiments of the present invention.

### BEST MODE FOR CARRYING OUT THE INVENTION

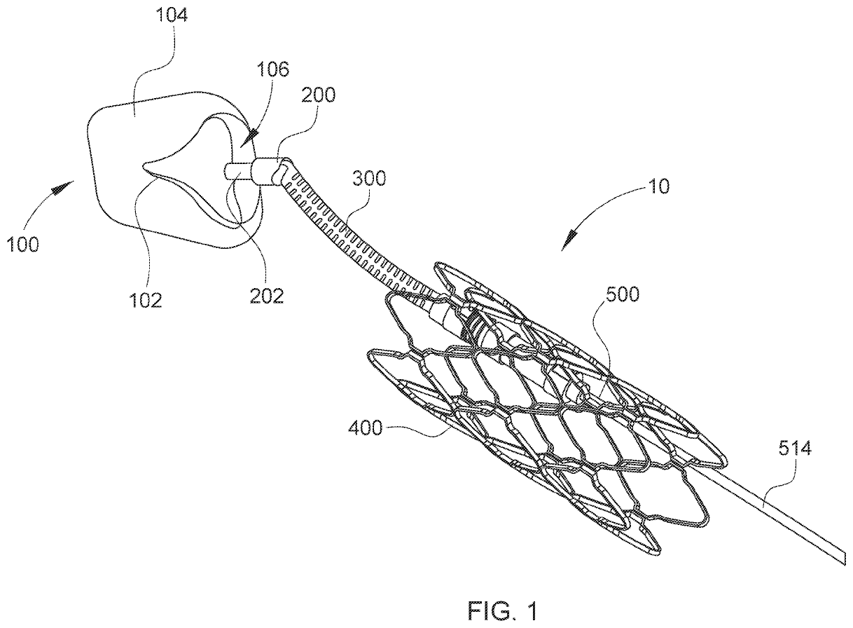

Referring first to FIG. 1, the inventive coaptation device delivery system of the present invention 10, as described herein, is best understood in relation to the prosthesis it is configured to deliver. Thus, the narrative herein includes, in the first instance, a description of the TV prosthesis 100, referred to herein as a coaptation sail, that is positioned and placed by the delivery system. FIG. 1 shows that in embodiments the coaptation sail 100 includes internal 3D-shaped nitinol wires 102 at least partly enclosed and covered in various porous and non-porous materials 104. It includes a proximal medial section 106 between the nitinol wires. The coaptation sail 100 extends somewhat centrally into the TV to provide a coaptation surface for the native TV leaflets.

At the proximal medial section of the coaptation sail, a gimbal/coupler subassembly 200 connects the coaptation sail 100 to a nitinol steering tube subassembly 300. The gimbal 202 connects the coupler 204 to the coaptation sail 100 and provides multi-axial rotation of the coaptation sail, relative to the coupler 204, within the TV annulus. The coupler 204 includes a proximal portion 204a and a distal portion 204b, which capture the gimbal in a way such that the gimbal includes degrees of freedom via rotational and swivel motions relative to the coupler. The coupler is connected to the distal end 300a of the steering tube with pins 300b. And the nitinol wire frame 102 of the coaptation sail 100 is connected to the distal end of the gimbal shaft 202a gimbal with a coupling clamp 202b.

The steering tube 300 and associated tensioner rod subassembly 500 is attached to the stent 400 and the delivery system handle to provide multi-axial adjustable positioning of the coaptation sail 100. Upon tensioning and/or rotation of the steering tube subassembly within the stent 400, the coaptation sail is positioned and aligned with the TV annulus engaging the native TV leaflets to treat a wide variety of anatomies. The IVC stent 400 is constructed from nitinol and is positioned in the IVC, near the juncture of the right atrium (RA) and IVC, and it provides anchoring of the tricuspid valve prosthesis in the IVC. Note that the anti-thrombogenic covering on the steering tube 300 is not shown.

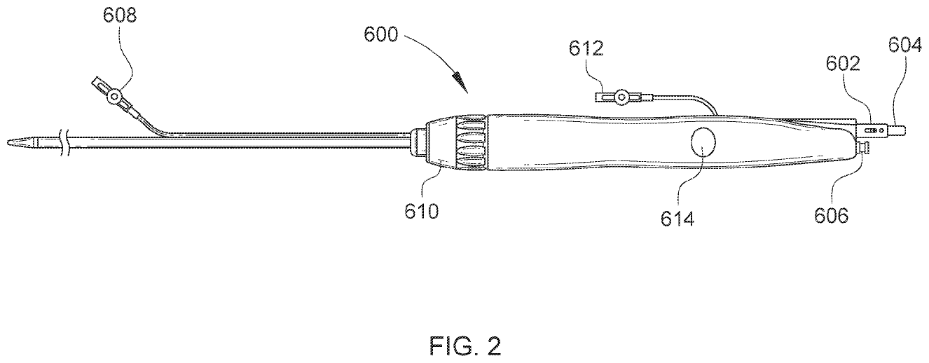

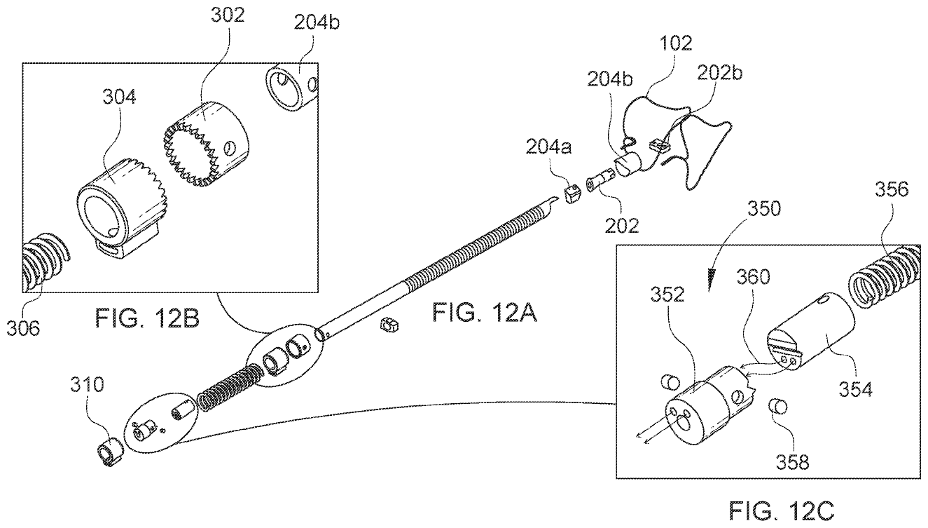

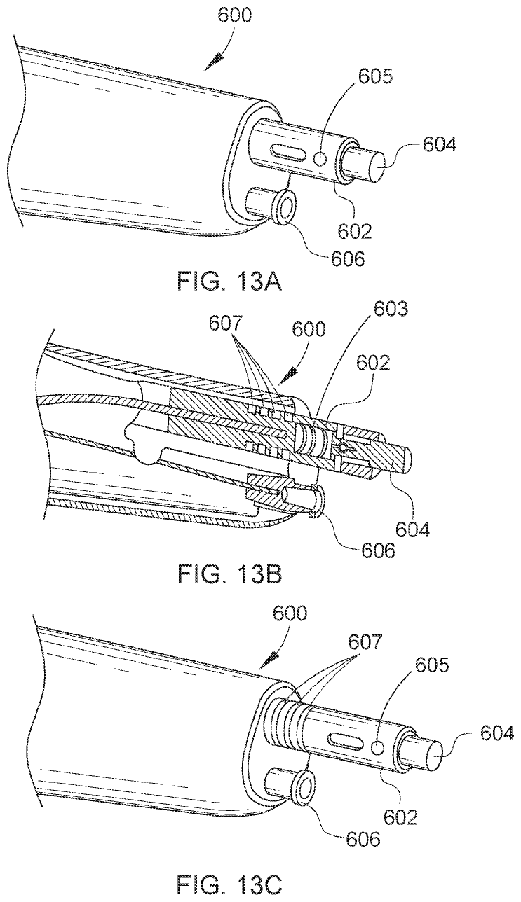

The main components of the delivery system are illustrated in FIG. 2 and FIGS. 4-13. In anticipation of further disclosure (see the narrative relating to FIGS. 13A-13C, below), and referring now to both FIG. 2 and FIGS. 13A-13C, the system at its proximal end includes a prosthesis delivery system handle 600, which provides multiple functions during the preparation and implantation of tricuspid valve prosthesis and provides a controlled implantation allowing retrieval, if needed. The control handle includes a tension knob 602, a release knob 604, and a guide wire lumen and luer lock 606. A flush-port 608 allows flushing the system with heparinized saline to remove all air from the inner catheter and prosthesis. A sheath dial 610 is operatively connected to a delivery sheath and retracts the outer sheath upon rotation to slowly expose the coaptation prosthesis. A heparinized saline drip line 612 promotes non-coagulation of the adjustment mechanisms during prosthesis delivery. A stent release button 614 prevents accidental prosthesis release until pushed by preventing the outer sheath from fully retracting. The tension dial knob 602 adjusts the amount of tension applied to the prosthesis upon rotation, and the release knob button 604 disconnects the delivery system from the prosthesis.

The TR patient population includes many anatomical variations beyond the basic dimensions such as IVC diameter and TV annulus size. The orientation of the IVC ostium (IVC ostial plane), and the distance to TV annulus as well as the TV annulus orientation (TV annulus plane), introduce additional challenges in positioning the coaptation sail. Yet, the orientation and position of the coaptation sail in the 3D volume of the RA and TV annulus is crucial to successful TR reduction. As such, additional prosthesis and delivery system capabilities are required to ensure the inventive coaptation prosthesis and delivery system is able to treat the wide variety of TR patient population anatomies.

The following novel system provides the needed capabilities, with features and functions that enable the inventive coaptation prosthesis and delivery system to treat more diverse TR patient anatomies. Each of the capabilities provides advantages either individually or in combination. Notably, several novel aspects of the coaptation sail 100 and gimbal 202 and coupler 204 are disclosed co-pending International Patent Application, which shares the inventors of the present invention and is filed concurrently herewith, said application entitled “Coaptation Device”, incorporated in its entirety herein by reference.

The prosthesis and delivery system elements included in this disclosure include as principal components: (1) a novel gimbal design fabricated from medically suitable materials, such as polyether ether ketone (PEEK), stainless steel, titanium, etc., (2) a steering tube (with multi-axis adjustability, and fabricated from materials such as nitinol, PEEK, etc.; (3) a stent design for steering tube attachment, also fabricated from the same materials; and (4) a tensioning rod subassembly, fabricated from PEEK, stainless steel, titanium, polyimide, and etc.

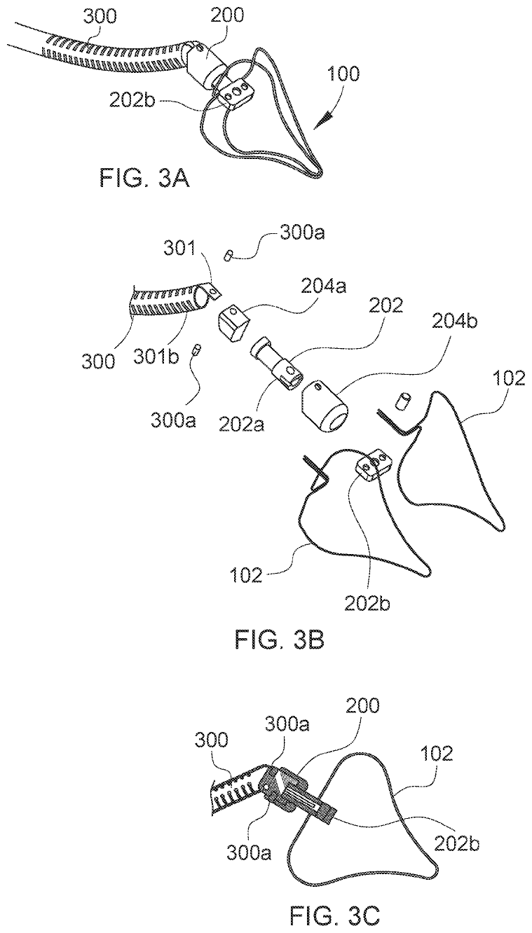

Gimbal 202 connects the coupler 204 to the coaptation sail 100 and enables multi-axial movement of the coaptation sail relative to the coupler. The ability of the coaptation sail to self-orient within the TV annulus ensures that it does not impinge on the native leaflets, causing more TR, but instead self-aligns with the coaptation commissures to increase native leaflet coaptation. Several views of the gimbal and coupler assembly are shown in FIGS. 3A-3C to illustrate the design elements. The porous and non-porous covering materials including a middle section between the nitinol wires or the porous and non-porous covering between the outer layer and nitinol wires are not shown.

FIGS. 3A-3C are perspective views showing the steering tube 300 attached to the gimbal 202 and coupler 204 including the nitinol wires 102. The exploded view of FIG. 3B illustrates the different components of the assembly including the gimbal 202 and coupler 204. Note the angled tab 301 on the distal end 301b of the nitinol steering tube 300. During sheathing of the prosthesis (the coaptation sail), tab 301 is generally aligned with the axis of the steering tube; whereas upon unsheathing it flexes inwardly into the position shown. This is due to the spring property of the tab material.

The cross-sectional view of FIG. 3C illustrates the attachment of the coupler 204 to the steering tube 300 via pins 300a. Note that the gimbal is contained within, captured by, and extends through the proximal and distal portions of the coupler, 204a, 204b, respectively.

The purpose of the steering tube 300 is to position the coaptation sail 100 relative to the TV annulus. The multi-axis adjustability of this design, flexure in several planes, and rotation relative to the stent, collectively enable the position of the coaptation sail to be finely tuned to a patient's anatomy.

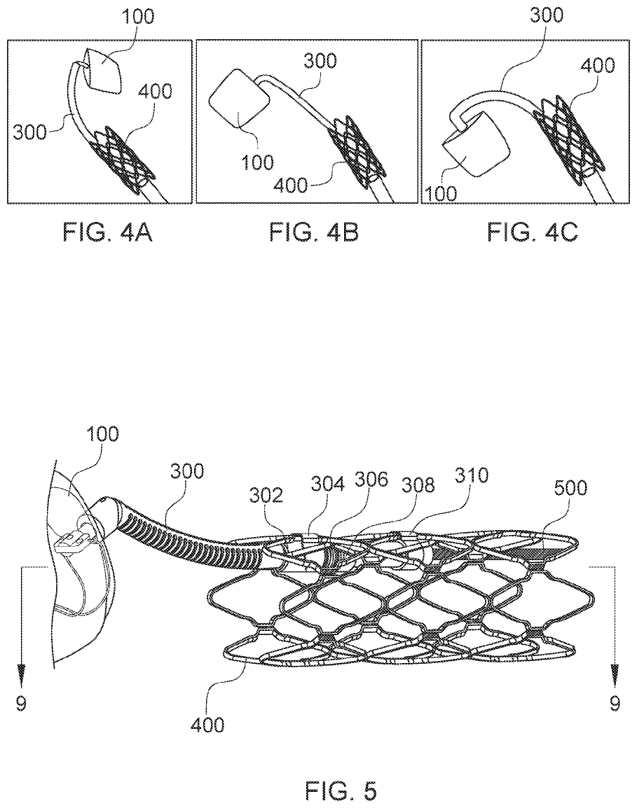

The schematic views of FIGS. 4A-4C depict several orientations of one steering system at various flexure amounts and rotations. Note that steering tube flexure is a result of the threaded insert (interacts with the tension rod subassembly) being rotated to increase the tension in the tensioning member which effectively shortens whichever side of the steering tube (where material is removed) to create curvature. The steering tube material is typically nitinol, but other materials (PEEK, stainless steel, etc.) are also suitable.

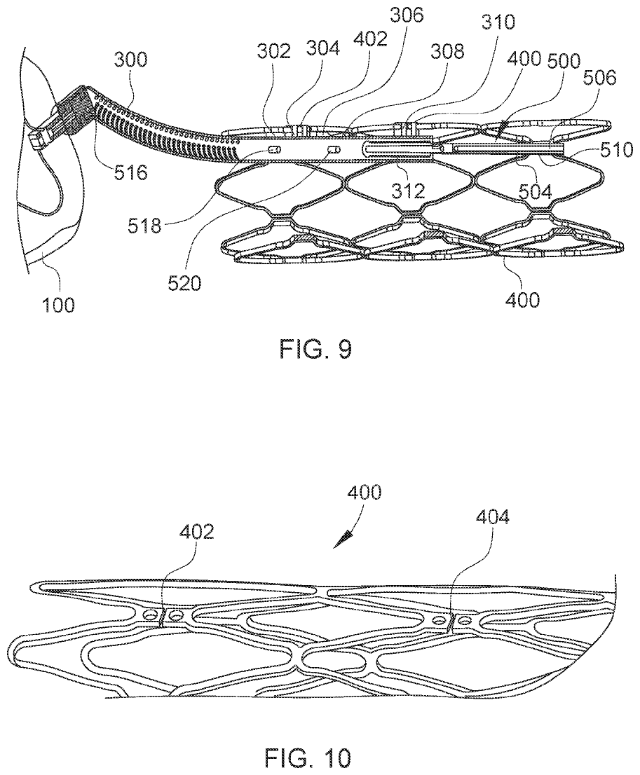

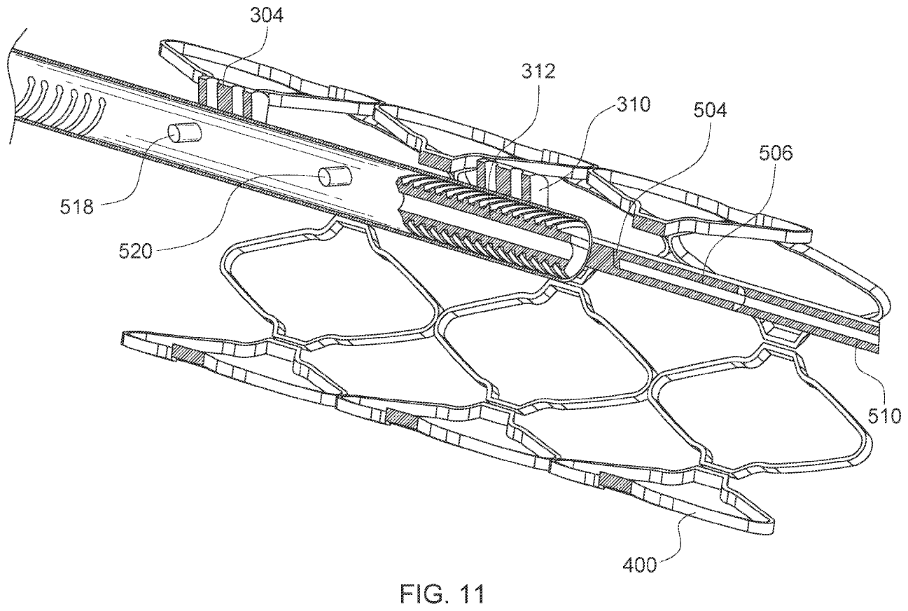

One configuration of the steering system subassembly is shown assembled in FIG. 5. The illustrations in FIG. 9 and FIG. 11 are cross-sectional views of the steering configuration of FIG. 5 and further include details of the tensioning rod subassembly coupled to the steering rod subassembly.

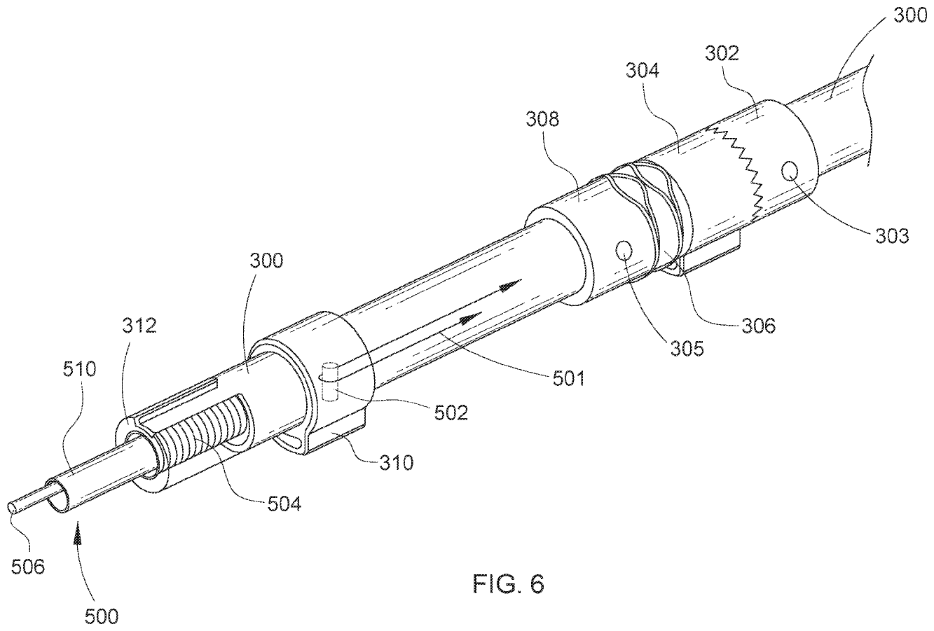

FIG. 6 is an upper perspective view showing the components comprising the steering system and tensioning rod subassemblies, here also illustrating how the tensioning member 501 (i.e., suture thread) wraps around a suture pin 502 (i.e., an anchor pin). Note that as the threaded component 504 rotates to create tension, it imparts little to no moment to the tensioning member 501, and the axial load on the threaded components effectively locks it into position, as no counter torque is present for unthreading.

The individual components and respective functions shown in FIGS. 5-12 are the steering tube 300, as described above. It is to be understood that based upon the as-cut pattern (and additional cross-through pins), the flexure may occur in several different directions. A serrated collar 302 is affixed to the steering tube 300 using a cross-through pin 303, which provides serrations on the inboard/proximal end and thereby locks the steering tube rotation angle to a serrated stent collar 304 having serrations that interdigitate and mate with those of the serrated collar. This provides flexure direction via tensioning member routing below cross through pin (see esp. FIG. 9). The serrated stent collar 304 attaches the steering tube 300 to the stent 400 while allowing steering tube rotation relative to the stent.

A compression spring 306 provides spring force to engage the serrations of serrated collar 302 to serrated distal (first) stent collar 304 while allowing manual rotation of the steering tube relative to the stent. In embodiments, the compression spring 306 may be internal (not shown) to the steering tube to provide the locking spring force.

Although use of a serrated collar is shown, alternative embodiments include a tapered collet, which provides higher angular rotation resolution, or a cross pin and grooved collar arrangement, etc. An alternative embodiment (also not shown here) may enable compression of the spring, rotation of the steering tube, and locking the rotation angle via the delivery system handle controls.

A ring collar 308 attaches to the steering tube also using a cross-through pin 305 and counteracts the spring force of the compression spring 306. A proximal (second) stent collar 310 attaches to the steering tube and allows rotation and translation of the steering tube relative to the stent.

A tensioner rod subassembly 500 provides a secure connection between the prosthesis and the delivery system handle to deliver rotational (torque) forces to the steering tube through the tension rod and thereby to adjust tension for steering tube flexure.

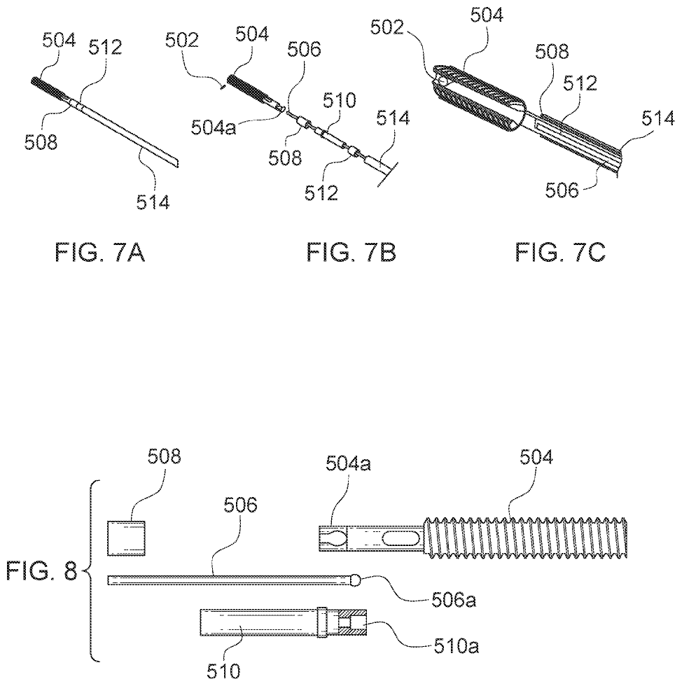

The tensioning rod subassembly illustrated in FIGS. 7A-7C is shown in three views: an isometric assembly view, an exploded perspective view, and a cross-sectional perspective view. The individual components and respective functions shown include a suture pin 502 (FIG. 7B) affixed to the threaded tensioner 504 and connected to the tensioning member 501. The threaded tensioner 504 component, when threaded in or out of the threaded insert 312, adjusts the tension in the tensioning member to provide flexure to the steering tube 300. Balled wire 506 is combined with the threaded tensioner 504 using an balled expansion 506a at the distal end of the balled wire, which is captured in and between shaped recesses 504a and 510a in the proximal end of the threaded tensioner and the distal end 510a of the tension interlock 510 encircled by a tension collar 508, to provide an interface that locks into position to provide a torque-able assembly while allowing disconnection when the tension interlock 510 is translated away from the threaded tensioner 504. The balled wire is connected at its proximal end to the release button 604 in the delivery system (see FIGS. 2 and 13A-13C). The tensioner collar 508 is affixed to cover the proximal end 504a of the threaded tensioner 504 and the distal end 510a of the tension interlock 510. The threaded tension interlock 510 is affixed to the tension tube 514 and the torsion tube 514.

A radiopaque band 512 is affixed over the tension tube 514 and provides fluoroscopic imaging aid in evaluating the relative position of the threaded tensioner 504 inside the threaded insert 312. The tension tube 514 is affixed to tension interlock 510 and connected to the tension knob 602 in the delivery system.

The interlock assembly's individual components are shown side-by-side in FIG. 10. Although this illustration shows the components set apart, the final assembly is co-axial in nature. Here also the tension interlock 510 has been rotated 180-degrees to illustrate the end feature that captures the balled end 506a of the balled wire 506. As noted, the expanded spherical end of the balled wire 506 fits into the pocket at the end of the threaded tensioner 504 and when the tension interlock 510 is positioned over the round end, along with the tensioner collar 508 over all components at the interface, the balled wire is completely captured. Both the threaded tensioner 504 and tension interlock 510 have a “D” shaped end that when together are contained within the tensioner collar 508. This interlock assembly provides an interface that locks into position to provide a torque-able assembly while allowing disconnection when the tension interlock 510 is translated away from the threaded tensioner 504.

Although this configuration described a “captured balled wire in a pocket”, this is not limiting: alternatives include an L-shaped wire end that fits into either an L-shaped pocket in either side of the D-shaped ends or a slot with a hole in the end for the L-shaped wire end. Each alternative would require a tensioner collar to constrain the joint until disconnection is desired.

A cross-sectional view of the steering system and tensioning rod subassemblies is shown in FIGS. 9 and 11. Note the routing of the tensioning member (i.e., suture, etc.) in this configuration is from a distal end 516, under medial point 518, to proximal return 520, and back to 516 along the same route. The typical assembly method entails routing the tensioning member inside a protective lubricious tube (i.e., FEP, PTFE, etc.) that loops around pin 520 and ties off at 516 while passing under cross-through pins 518. The protective lubricious tubing (not shown) prevents damage to the tensioning member from the inside edges of the steering tube 300 during flexure or natural prosthesis movement in the clinical setting. Additional cross through pins may be distributed throughout the length of the steering tube to create additional pivot points which, when combined with various laser-cut patterns, provide multi-directional steering tube flexure.

The tensioning member path over/under or from one side to the other of each cross through pin may vary according to the flexure desired. Additional guides may be placed on the cross through pins controlling the path of the tensioning member. Additional tensioning members my connect to these cross-through pins to enable various amounts of force applied to different sections of the steering tube through the use of co-axial or non-co-axial threaded inserts and tensioning rod configurations (not shown).

The attachment of the threaded insert to the steering tube, wherein the steering tube has “T” shaped features that interlock with the threaded insert, provides securement without fasteners or adhesives.

As can be seen in FIGS. 9-10, the stent 400 is configured for attachment to the steering tube. Serrated stent collar 304 and stent collar 310 each include two pins passing through the collars into aligned holes 402, 404 in the stent (see FIG. 10) for attachment of the steering tube to the stent. A stent strut gap between holes allows the one-piece stent collars to be securely captured on the stent strut.

A single co-axial tensioning rod subassembly is shown in FIG. 11. As may be surmised thus far, the purpose of the tensioning rod subassembly is to adjust the flexure of the steering tube and once positioned, to fixate the amount of tension, then disconnect from the prosthesis upon completion of the implantation procedure. The tensioning rod subassembly is detailed in each of FIGS. 7A-9, 11-12. The connection between the steering system subassembly and the tensioning rod subassembly is achieved through the threaded insert 312, attached to the steering tube and threaded to threadably connected with a threaded tensioner 504.

The proximal end of the delivery system, as it relates to the tensioning rod subassembly is shown in FIGS. 13A-13C. Note the delivery system handle 600, tension knob 602, release knob 604, and guide wire lumen and luer lock 606. The cross-sectional view (FIG. 13B) illustrates how each component is structurally and operationally related and how the compression spring inside the tension knob 602 that applies spring force to keep the interlock assembly connected. A side set screw 605 is included for safety to ensure the two components remain connected. When ready to disconnect, the side set screw is loosened allowing the tension knob 602 to be retracted for disconnection from the prosthesis.

Extended view (FIG. 13C) illustrates white ring visual indicators 607 that provide an applied tension reference point. Additional delivery system handle configurations (not shown) include multiple dials to either rotate and/or flex the steering tube, levers for locking or unlocking the coaptation device's position, and other control and actuation mechanisms for multi-directional movements of the coaptation device to ensure very precise positioning and placement in the TV. The implantation procedure using the coaptation device and its delivery system resembles other transcatheter procedures using fluoroscopic and echogenic visualization and includes the following steps:

First, the femoral vein is accessed and an anatomical and TVR assessment is performed. Next, the coaptive prosthesis is prepared and sheathed and system preparation is verified. The cardiac guide wire is inserted through the distal tip (i.e., the nosecone) of the control handle and passed through to an exit at the proximal luer lock near the release knob. After that, a heparinized saline pressure bag is connected to the side stopcock of the delivery system handle and the bag pressure is set accordingly to ensure slight flow through sheath tip. The prosthesis is loaded in the percutaneous delivery system, in embodiments a 0.035 in 0.89 mm nitrex/nitinol/stainless steel guide wire compatible system.

The physician/operator next advances the coaptation prosthesis and its control mechanism over the guide wire through the access site into the right atrium, using image guidance. The physician/operator will then observe the radiopaque nosecone and outer sheath tip marker using fluoroscopy.

To deploy the coaptation prosthesis, the delivery handle is pinned to a surface, and the sheath dial is rotated (CW), such that the tip of the outer sheath retracts and gradually exposes the sail into the right atrium, during which an outer sheath slides through the introducer sheath. The sheath dial rotation is stopped when the coaptation sail and the steering tube are entirely unsheathed. At this point, an assessment is made as to the coaptation sail position in relation to the TV annulus and its interaction with the native leaflets.

The sail is repositioned as needed for optimal results, either by: (1) advancing, retracting or rotating the entire prosthetic system; (2) further rotating the sheath dial (CW) to expose more of the prosthesis; or (3) rotating the tension knob (CCW) to flex the distal portion of the stent, with due caution taken to ensure this this action is taken only the stent is exposed.

Changes in regurgitation and valve function may then be assessed with ultrasound imaging (ICE, TTE).

Prosthesis Deployment: To deploy the prosthesis, the physician/operator carefully rotates the sheath dial 610 (CW) until it stops to expose the stent while maintaining the position of the distal edge of stent in the IVC. Note that the stent remains constrained in the sheath at its proximal end, and the stent is in apposition in the IVC during expansion.

Using a hex key provided in sterile prep materials, the operator carefully loosens the set screw 605 that connects the tension knob 602 to the release button 604. To disconnect the delivery system from the stent, the operator provides a gentle push/pull action with the tension knob and release button—the tension knob providing the pull, and the release button providing a push. The result is carefully observed and disconnection is verified by carefully pulling the tension knob with the release button away from the handle 600.

To completely release the stent in the IVC, a push and hold of the release button on the delivery system handle is employed, followed by rotation of the sheath dial (CW) until the stent is fully expanded in the IVC.

Note that the order of some steps may be reversed if appropriate under the circumstances.

Next, the operator must verify that the delivery system is fully detached from the prosthesis by gently advancing/retracting the delivery system.

The sheath dial is then rotated (CCW) to advance the sheath to the nosecone.

Finally, the guide wire is removed prior to the stent release, and it is removed from the now implanted and positioned prosthetic system.

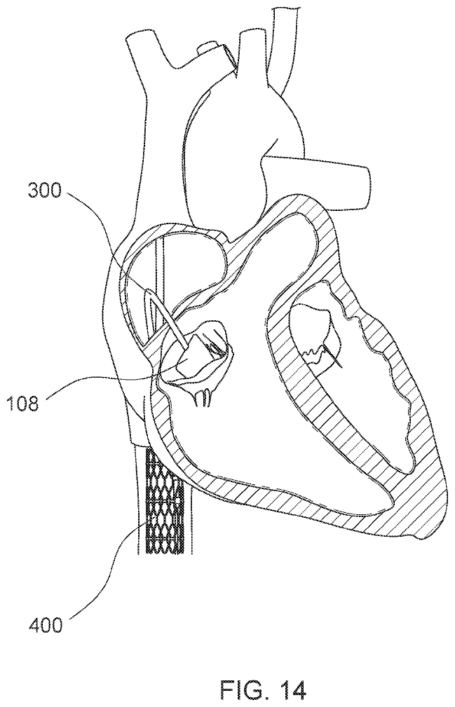

FIG. 14 is a highly schematic cross-sectional view of the coaptation sail 108 aligned and deployed in the TV and the anchoring stent 400 in the IVC, the two components connected by the steering tube 300, all in accordance with embodiments of the present invention.

The foregoing description is directed to preferred embodiments of the invention, including the best mode for carrying out the invention currently contemplated by the inventors. However, these do not exhaust or even begin to exhaust possible alternative embodiments, whether those are found in substantially equivalent alternative structures or substantially equivalent alternative operations, or both. The embodiments are, instead, described and presented for illustrative purposes, while it will be understood by those with skill in the art that this is not desired to limit the invention to the exact construction, dimensional relationships, and operation shown and described. Various modifications, alternative constructions, changes and equivalents will readily occur to those skilled in the art and may be employed, as suitable, without departing from the true spirit and scope of the invention. Such changes might involve alternative materials, components, structural arrangements, sizes, shapes, forms, functions, operational features, alternative order of method steps or the like. Therefore, the above description and illustrations should not be construed as limiting the scope of the invention, which is defined by the appended claims.

What is claimed as invention is:

1. A system for treating tricuspid valve regurgitation (TVR), comprising:
a stent configured for implantation in the inferior vena cava (IVC) proximate the juncture with the right atrium (RA);
a swivel apparatus tethered to said stent;
a coaptation member connected to said swivel apparatus, said coaptation member configured for percutaneous delivery to the RA for deployment within the tricuspid valve (TV) annulus;
a steering system including a steering tube having a proximal end and a distal end, said steering tube interposed between said stent and said swivel apparatus, said steering system configured to enable multi-directional positioning and placement of said coaptation member before and after said stent is implanted in the IVC; and
a control handle for an operator use to percutaneously guide said stent to the IVC and said coaptation member to the RA and to provide inputs to said steering system during a prosthesis implantation procedure;
wherein when said stent is implanted in said IVC and said coaptation member is positioned within the RA, said swivel apparatus enables said coaptation member to auto-rotate in relation to the TV annulus to optimize coaptation with native leaflets, and said control handle enables an operator to flex and rotate said steering tube to finely position said coaptation member in relation to the TV.

2. The system of claim 1, wherein said swivel apparatus is a coupler/gimbal assembly.

3. The system of claim 2, wherein said coupler/gimbal assembly includes a coupler having a proximal portion pivotally connected to said distal end of said steering tube, a distal portion pivotally connected to said proximal portion, said distal portion having a cylindrical through passage, a gimbal having a head captured between said proximal and distal portions of said coupler, and a cylindrical shaft inserted through said through passage, said gimbal shaft having a distal end configured to attach to said coaptation member.

4. The system of claim 3, wherein said head of said gimbal and said distal portion of said coupler are configured to enable said gimbal to pivot and rotate in relation to said coupler.

5. The system of claim 4, wherein said coupler/gimbal assembly is configured to provide multi-axial rotation of said coaptation member relative to said coupler within the TV annulus.

6. The system of claim 1, wherein said steering tube remains in place, connecting said stent and said swivel apparatus after deployment of said coaptation member.

7. The system of claim 6, wherein said stent is connected to said steering tube with at least two stent collars that lock said steering tube rotation but selectively permit manual rotation of said steering tube by said operator during an implantation procedure.

8. The system of claim 1, wherein said steering tube is operatively connected to said control handle.

9. The system of claim 8, wherein said steering tube and said control handle are collectively configured to enable control inputs through said control handle by the operator to flex and rotate said steering tube after said stent is anchored in the IVC.

10. The system of claim 9, wherein flexure in said steering tube is controlled by the as-cut pattern in manufacture and by one or more cross-thru pins disposed in said steering tube.

11. The system of claim 10, wherein said steering tube includes a plurality of cross-thru pins about which a tensioning member is routed and operatively connected to said control handle, whereby inputs through the control handle to said tensioning member induce flexure in said steering tube.

12. The system of claim 11, including a first cross-thru pin in a proximal portion of said steering tube, a second cross-thru pin distal relative to said first cross-thru pin, and an eyelet in said distal end of said steering tube, wherein said tensioning member is connected internally at said eyelet, and wherein inputs that pull on said tensioning member cause said steering tube to flex.

13. The system of claim 12, further including:
a steering subassembly slidably disposed in said proximal end of said steering tube and threadably connected to a tensioning rod subassembly interposed between said steering tube and said control handle, said tensioning rod subassembly including a tension tube connected to said control handle;
wherein said first cross-thru pin is disposed within said steering tube subassembly, whereby operator inputs pulling on said tension tube transmit a pulling force on said steering tube subassembly and thereby cause flexure in said steering tube.

14. The system of claim 1, further including an angled tab made of shape memory alloy and disposed on said distal end of said steering tube, wherein during sheathing of said coaptation member and said coupler/gimbal assembly, said angled tab is generally aligned with the axis of the steering tube, and whereupon unsheathing said coupler/gimbal assembly enables said angled tab to bend inwardly due to the spring property of the angled tab material.
